(19) United States
(12) Patent Application Publication
(10) Pub. No.: US 2025/0017600 A1
(43) Pub. Date: Jan. 16, 2025

(54) PREPARING A TIBIA FOR RECEIVING TIBIAL IMPLANT COMPONENT OF A REPLACEMENT ANKLE

(71) Applicant: WRIGHT MEDICAL TECHNOLOGY, INC., Memphis, TN (US)

(72) Inventors: Meghan KUBACKI, Memphis, TN (US); Ramon LUNA, Arlington, TN (US); Terrance W. STROHKIRCH, Memphis, TN (US)

(73) Assignee: WRIGHT MEDICAL TECHNOLOGY, INC., Memphis, TN (US)

(21) Appl. No.: 18/813,095

(22) Filed: Aug. 23, 2024

Related U.S. Application Data
(62) Division of application No. 17/424,416, filed on Jul. 20, 2021, now Pat. No. 12,108,959.
(60) Provisional application No. 62/853,818, filed on May 29, 2019.

(51) Int. Cl. A61B 17/16; A61B 17/17; A61B 17/56; A61F 2/42; A61F 2/46

(52) U.S. Cl. CPC A61B 17/1682; A61B 17/1717; A61B 17/1775; A61F 2/4606; A61B 2017/564; A61F 2002/4205; A61F 2002/4207

(57) ABSTRACT

Disclosed is are various methods of preparing a tibial intramedullary canal for receiving a tibial implant, methods of preparing a talus for receiving a talar implant, and some examples of a power driver adapter that can be used to carry out the preparation of the tibial intramedullary canal.

### CROSS-REFERENCE TO RELATED APPLICATIONS

This application is a divisional of U.S. patent application Ser. No. 17/424,416, filed on Jul. 20, 2021, which is a National Stage Application, filed under 35 U.S.C. 371, of International Patent Application No. PCT/US2020/015373, filed on Jan. 28, 2020, which claims priority to U.S. Provisional Patent Application No. 62/853,818 filed May 29, 2019, the entireties of which are incorporated herein by reference

### FIELD OF DISCLOSURE

The present disclosure relates to an ankle replacement procedure.

### BACKGROUND

An ankle joint may become severely damaged and painful due to arthritis from prior ankle surgery, bone fracture, infection, osteoarthritis, post-traumatic osteoarthritis or rheumatoid arthritis, for example. Options for treating the injured ankle have included anti-inflammatory and pain medications, braces, physical therapy, amputation, joint arthrodesis, and total ankle replacement.

Current ankle joint replacement options include preparing the distal end of the tibia by drilling through the calcaneus and the talus from the bottom of the foot to access the distal end of the tibia to ream the tibial intramedullary canal. Such approaches require an additional incision in the heel. The patient's recovery time can be extended and can delay the weight-bearing time after the surgery.

A recent improved ankle joint replacement procedure involves approaching the ankle joint space with a broach from the anterior side and preparing the intramedullary canal of the tibia manually.

### SUMMARY

Disclosed is a method of preparing an intramedullary canal in a tibia for receiving a tibial implant. In some embodiments, the method comprises (a) resecting the distal end of the tibia and forming a resected joint space for the tibial implant, wherein the joint space comprises a tibial resection surface at the distal end of the tibia and is open at the anterior side; (b) positioning a power driver, equipped with a cutting tool bit, into the joint space from the anterior side, wherein the cutting tool bit is aimed toward the intramedullary canal of the tibia; and (c) cutting into the intramedullary canal using the power driver to form a tibial hole or a tibial cavity. The power driver can be used in conjunction with a guide assembly that can assist alignment of the cutting tool bit.

According to another aspect of the present disclosure, the method of preparing an intramedullary canal in a tibia for receiving a tibial implant can involve positioning the power driver, equipped with a cutting tool bit, into the joint space from the posterior side.

According to another aspect of the present disclosure, the method of preparing an intramedullary canal in a tibia for receiving a tibial implant can involve positioning the power driver, equipped with a cutting tool bit, into the joint space from the lateral side.

Also disclosed is a power driver adapter configured for cutting into an intramedullary canal of a tibia. The power driver adapter comprises an elongated body having a driving end, a cutting tool bit receiving end, and a longitudinal axis. The driving end comprises a drive shaft coaxially located with the longitudinal axis and is configured to mate with a power delivering unit that rotates the drive shaft coaxially about the longitudinal axis. The cutting tool bit receiving end comprises a cutting tool bit receiving base that is configured for engaging with a cutting tool bit and rotates the cutting tool bit for cutting action, where the cutting tool receiving base rotates with a rotational axis that is orthogonal to the longitudinal axis of the elongated body and translates superiorly and inferiorly. The elongated body comprises a series of gears connecting the drive shaft to the cutting tool bit receiving end. The series of gears are configured in an arrangement that converts the coaxial rotation of the drive shaft to the rotation and translation of the cutting tool bit receiving base.

A surgical instrument kit that includes the power driver adapter is also disclosed.

### BRIEF DESCRIPTION OF THE DRAWINGS

The inventive concepts of the present disclosure will be described in more detail in conjunction with the following drawing figures. The structures in the drawing figures are illustrated schematically and are not intended to show actual dimensions.

FIG. 1 is an illustration of an ankle joint.

FIG. 2 is a representation of a resected joint space at the distal end of a tibia for receiving a tibial ankle replacement implant.

FIG. 3 is an illustration of the inventive power driver assembly of the present disclosure engaging the resected joint space of a tibia from the anterior side at the completion of the tibial preparation procedure.

FIG. 4 is another illustration of the inventive power driver assembly of FIG. 3 seen from a different angle.

FIG. 5 is an illustration of the power driver adapter according to the present disclosure.

FIG. 6 is an illustration of the guide assembly installed in the resected joint space.

FIG. 7 is an illustration of the inventive power driver assembly of the present disclosure being positioned in the resected joint space of a tibia from the posterior side.

FIG. 8 is an illustration of the inventive power driver assembly of the present disclosure being positioned in the resected joint space of a tibia from the lateral side.

FIG. 9A is a side view illustration of the power driver of the present disclosure.

FIG. 9B is another side view illustration of the power driver of the present disclosure, similar to FIG. 9A.

FIG. 10A is a top-down view illustration of the power driver of FIG. 9A.

FIG. 10B is a top-down view illustration of the power driver of FIG. 9B.

FIG. 11A is a cross-sectional view of the power driver taken through the line A-A shown in FIG. 10A.

FIG. 11B is another cross-sectional view of the power driver taken through the line B-B shown in FIG. 10B in which the cutting tool bit receiving portion is in a START position.

FIG. 11C is a cross-sectional view of the power driver taken through the line C-C shown in FIG. 9B.

FIGS. 12A-121 are flowcharts illustrating the various embodiments of the methods for preparing the intramedullary canal of a tibia according to the present disclosure.

FIG. 13A-13C show an embodiment of the power driver adapter of the present disclosure having alignment arms for use in the environment.

FIG. 13D is an illustration of the inventive power driver of the present disclosure being positioned in the resected joint space of a tibia from the anterior side and oriented with the driver bit toward the talus bone.

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

Unless otherwise expressly stated, it is in no way intended that any method set forth herein be construed as requiring that its steps be performed in a specific order, nor that with any apparatus, specific orientations be required, unless specified as such. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

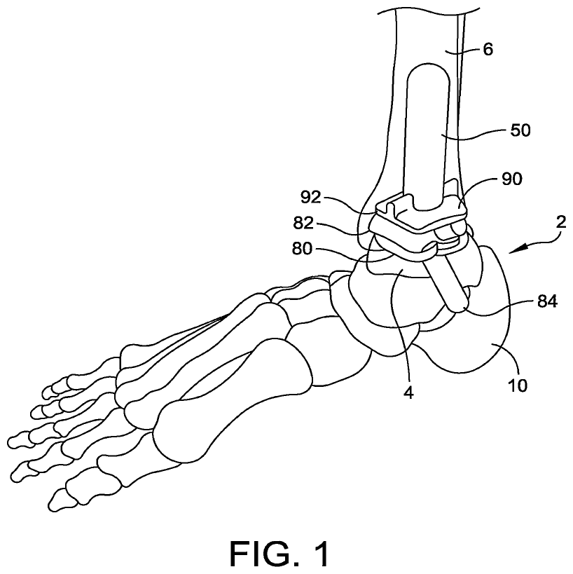

FIG. 1 illustrates an anatomic view of an ankle joint 2. The ankle joint 2 comprises a talus 4 in contact with a tibia 6 and a fibula (not labelled). A calcaneus 10 is located adjacent to the talus 4. In total ankle replacements, the talus 4 and the tibia 6 may be resected, or cut, to allow insertion of a talar implant and a tibial implant.

A total ankle replacement system can include a talar implant 80 and a tibial implant 90. The talar implant 80 can include an articulation surface 82 configured to mimic a natural articulation surface of the talus 4. The talar implant 80 can have a stem 84 that extends into the talus 4 to anchor the talar implant 80. A tibial implant 90 can be sized and configured for installation into the tibia 6. The tibial implant 90 can include a body comprising an articulation surface 92 configured to mimic a natural articulation of the tibia 6 and a tibial stem 50 extending into the intramedullary canal of the tibia 6 to anchor the tibial implant 90. The articulation surfaces 82, 92 of the respective implants 80, 90 replace the natural ankle joint surfaces, which are removed, to restore a range of motion that mimics the natural joint.

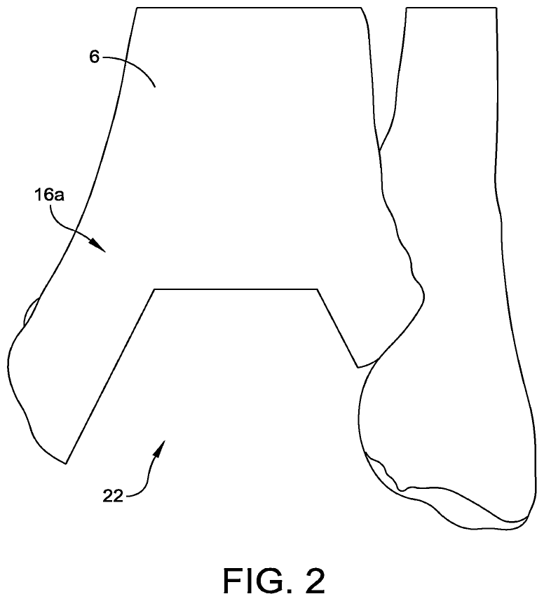

FIG. 2 is an illustration of a resected tibial end 16a of the tibia 6 in a human ankle showing the resected joint space 22.

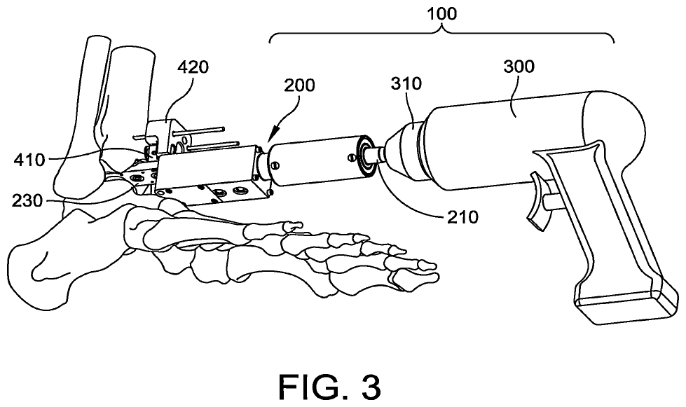

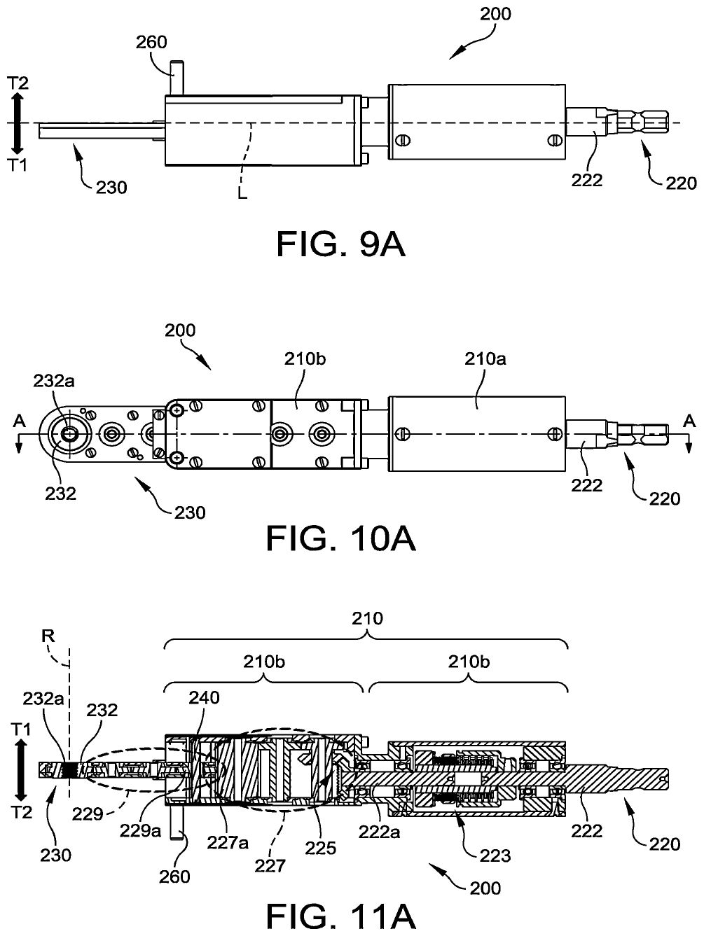

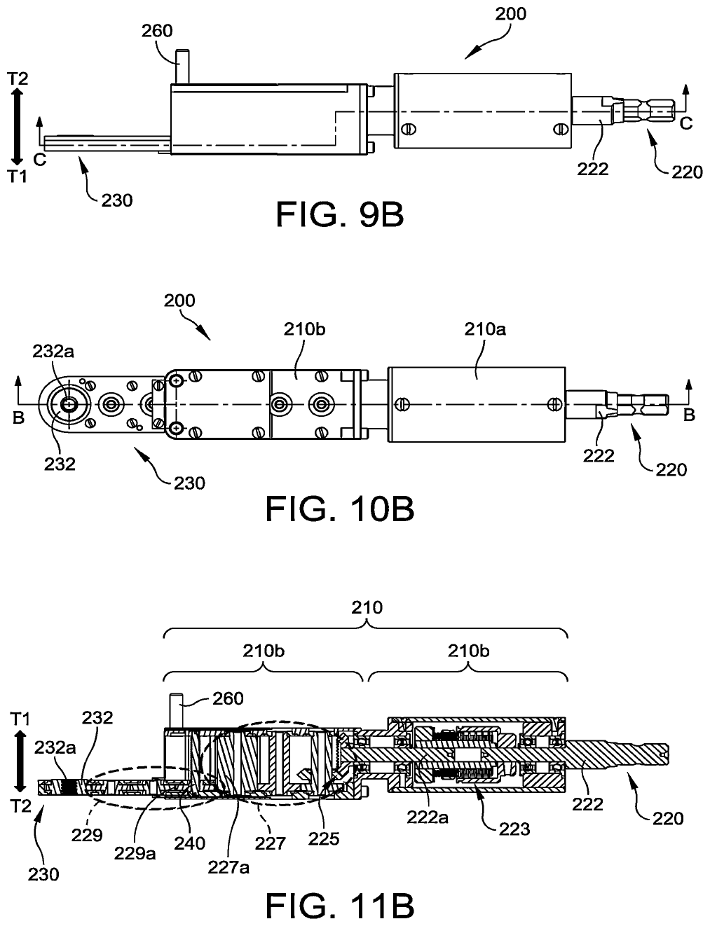

Referring to FIGS. 3-5, and 9-11, a power driver 100 configured for cutting into an intramedullary canal of a tibia is disclosed. The power driver 100 comprises a power driver unit 300 for hand-held operation and a power driver adapter 200.

Preferably, the power driver unit 300 is a handheld drill-like power tool that can rotatably drive the power driver adapter 200 and comprises a chuck 310 that engages the drive shaft 222 of the power driver adapter 200.

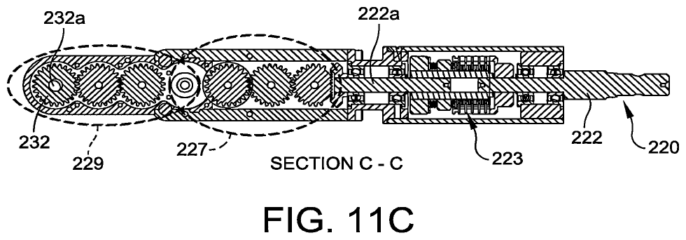

As shown in FIGS. 3-5, and 9-11, the power driver adapter 200 comprises an elongated body 210 having a first end 220, a second portion 230, and a longitudinal axis L. The first end 220 is configured as a driving end and has a drive shaft 222 coaxially located with the longitudinal axis L and configured to engage with the power driver unit 300 that rotates the drive shaft 222 coaxially about the longitudinal axis L. The second portion 230 is configured as a cutting tool bit receiving portion. The cutting tool bit receiving portion 230 is configured to engage with a cutting tool bit 500 and rotates the cutting tool bit 500 for a cutting action. For example, as shown in FIGS. 10 and 11, the cutting tool bit receiving portion 230 can be provided with a cutting tool bit receiving base 232 that is configured to receive the cutting tool bit 500 and securely hold the bit. The cutting tool bit receiving base 232 is configured to rotate about a rotational axis R that is orthogonal to the longitudinal axis L.

The cutting tool bit receiving base 232 in the illustrated embodiment is a disc-like piece that comprises a threaded hole 232a into which the cutting tool bit 500 can thread into. The cutting tool bit 500 that is configured for attaching to the cutting tool bit receiving base 232 comprises a threaded base stem (not shown). In some other embodiments, the cutting tool bit receiving base 232 can comprise a socket hole instead for receiving the cutting tool bit 500. The socket hole can have a square hole configuration or a hexagonal hole configuration and can have a spring-loaded detent system for holding the cutting tool bit 500. These are just examples and there are other suitable configurations for engaging the cutting tool bit receiving base 232 with the cutting tool bit 500 that would be readily understood by those of ordinary skill in the art.

The elongated body 210 comprises a first portion 210a and a second portion 210b. The elongated body 210 comprises a slip clutch in the first portion 210a and a series of gears in the second portion 210b that connect the drive shaft 222 to the cutting tool bit receiving portion 230. The series of gears are configured in an arrangement that converts the coaxial rotation of the drive shaft 222 to the rotation of the cutting tool bit receiving base 232. An example of such series of gears is shown in the cross-sectional view of the elongated body 210 in FIG. 11.

The first portion 210a comprises a drive shaft extension piece 222a that is connected to the drive shaft 222 by a slip-clutch mechanism 223. When the cutting tool bit receiving portion 230 reaches the bottom (the START) or the top (FINISH) position against the housing of the second portion 210b, the slip clutch 223 allows the drive shaft 222 to continue to be turned by the power driver 300 while the drive shaft extension piece 222a (and, in turn, the spur gears 227) stop turning. The second portion 210b comprises a group of spur gears 227 for transferring the rotational motion to the cutting tool bit receiving base 232. Between the group of spur gears 227 and the drive shaft 222a, a bevel gear arrangement 225 is provided to convert the coaxial rotation motion of the drive shaft 222, 222a, into the orthogonally oriented rotation motion of the cutting tool bit receiving base 232.

In some embodiments, the cutting tool bit receiving base 232 is a spur gear and the cutting tool receiving portion 230 can comprise one or more additional spur gears 229 that connects the cutting tool bit receiving base 232 with the group of spur gears 227. The cutting tool bit receiving base 232 and the additional spur gears in the group of spur gears 227 have a short or low profile and have a disc-like shape, which allows the cutting tool bit receiving portion 230 to maintain a low profile for positioning the cutting tool bit receiving portion 230 into the resected joint space 22. The last spur gear 227a among the group of spur gears 227 engages with the first spur gear 229a among the additional spur gears 229.

The cutting tool bit 500 can be one of many types of cutting tool bits that may be used in orthopedic procedures. In some embodiments of the power driver adapter 200, the cutting tool bit 500 is a reamer bit.

In some embodiments of the power driver adapter 200, the cutting tool bit receiving portion 230 can be configured to translate linearly along directions that are coaxial to the rotational axis R of the cutting tool bit 500 and orthogonal to the longitudinal axis L. The directions of the linear translation motion are illustrated by the arrows T1 and T2 in FIG. 11. The illustration of FIG. 11 is a sectional view seen from the side. Therefore, when the power driver adapter 200 is in operational use in the resected joint space 22, the arrow T1 represents the anatomical superior direction and the arrow T2 represents the anatomical inferior direction.

In some embodiments, in addition to the group of spur gears 227, the second portion 210b further comprises a helical thread arrangement that enables the linear translation of the cutting tool bit receiving portion 230. In the exemplary structure shown in FIG. 11, this helical thread arrangement comprises a helical threaded stem 240 and the first spur gear 229a among the additional spur gears 229 in the cutting tool bit receiving portion 230. The first spur gear 229a comprises a helical threaded hole in its rotational center that engages the helical threaded stem 240, which extends through the helical threaded hole. The helical threaded stem 240 does not rotate. When the cutting tool bit 500 is being rotated for a cutting action, as the first spur gear 229a rotates, its helical threaded hole cooperates with the helical thread on the helical threaded stem 240 and translates along the length of the helical threaded stem 240, thus, moving the whole cutting tool bit receiving portion 230 along the length of the helical threaded stem 240.

Depending on the rotational direction; the helical threads on the first spur gear 229a and the helical threaded stem 240 are appropriately handed (i.e., right handed or left handed) so that the first spur gear 229a, and hence the cutting tool bit receiving portion 230, translates in the direction T2 indicated by the arrow in FIG. 11 when the cutting tool bit 500 is being turned or driven in the cutting direction. Conversely, when the cutting procedure is completed, the power driving unit 300 is reversed and the cutting tool bit 500 is rotated in the opposite direction, the first spur gear 229a and the cutting tool receiving portion 230 will translate along the helical threaded stem 240 in the opposite direction T1.

When the power driver adapter 200 is used for reaming the distal end of a tibia, for example, the power driver adapter 200 is in position such that the cutting tool bit 500 is positioned and aimed toward the intramedullary canal of the tibia, the cutting tool receiving portion 230 would be in its START position, i.e., with the cutting tool receiving portion 230 at its most inferior position. This START configuration is shown in FIG. 11B. In FIG. 11B, one can tell that the cutting tool receiving portion 230 is in the START position because it is on the opposite side of the alignment pins 260. The function of the alignment pins 260 are described below in conjunction with FIGS. 3-5. When the power driver unit 300 is turned on to its cutting mode, as the cutting tool bit 500 turns in the cutting direction, the cutting tool holding portion 230 translates in the direction T2. This enables the cutting tool bit 500 to be driven into the intramedullary canal of the tibia without moving the whole power driver unit 300 and the power driver adapter 200. While the power driver unit 300 and the power driver adapter 200 assembly are being held stationary, the cutting tool bit 500 will automatically be driven into the intramedullary canal by the translation motion of the cutting tool bit receiving portion 230.

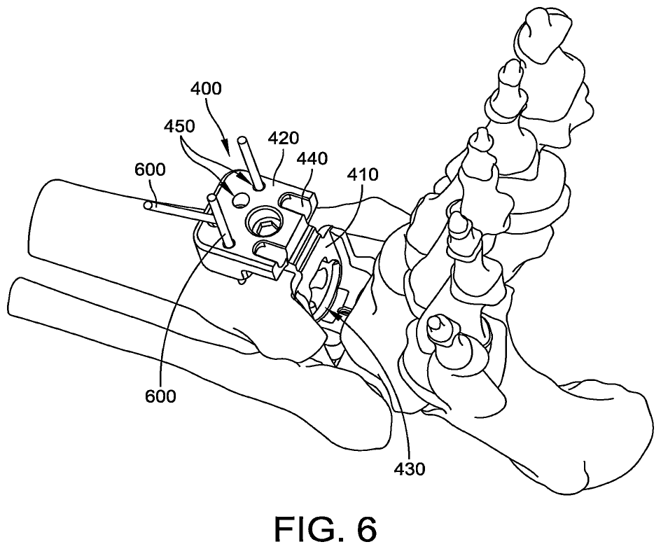

Referring to FIGS. 3, 4, and 6, in some embodiments, the power driver adapter 200 can be used in conjunction with a guide assembly 400 that can assist with positioning and alignment of the power driver adapter 200 in the resected joint space 22 during the procedure of preparing the intramedullary canal of the tibia. The guide assembly 400 comprises a guide portion 410 configured for attaching to the resected surface at a distal end of the tibia, wherein the guide portion comprises a hole 430 for receiving and allowing the cutting tool bit 500 to extend therethrough. The guide assembly 400 also includes a guide head portion 420 extending in the proximal direction from the guide portion 410 and configured for attaching to the anterior side of the tibia as shown in FIG. 6.

The power driver adapter 200 and the guide head portion 420 are configured to properly align and position the cutting tool bit 500 held in the power driver adapter 200 for cutting into the intramedullary canal of the tibia. The power driver adapter 200 can comprise of one or more alignment pins and the guide head portion 420 can comprise of one or more corresponding alignment slots for receiving the alignment pins to align the position of the power driver adapter 200.

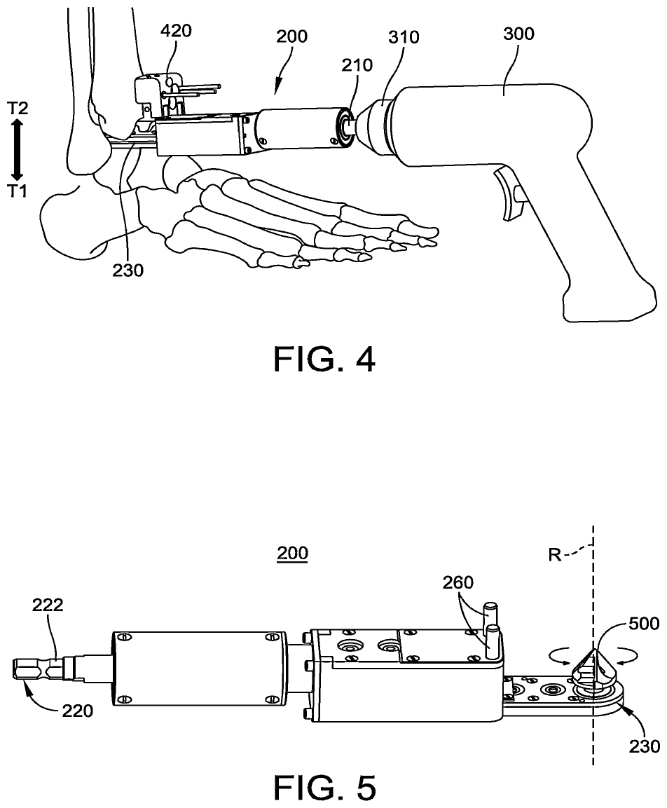

In the exemplary embodiment illustrated in FIGS. 4-6, the power driver adapter 200 comprises two alignment pins 260 and the guide head portion 420 comprises corresponding two alignment slots 440 for receiving the alignment pins 260. After inserting the cutting tool bit receiving portion 230 of the power driver adapter 200 into the joint space 22 from the anterior side, the two alignment pins 260 are inserted into the corresponding alignment slots 440 on the guide head portion 420. The power driver adapter 200 is oriented so that the cutting tool bit 500 is held in the cutting tool bit receiving portion 230 and positioned at the distal end of the resected tibia aimed toward the intramedullary canal of the tibia and ready to cut into the intramedullary canal of the tibia. In some embodiments, the alignment slots 440 are shaped and sized so that the alignment pins 260 and the alignment slots 440 establish a slip-fit engagement, which can help hold the power driver adapter 200 assembly securely in position during the cutting procedure. This arrangement would look very much like the ones illustrated in FIGS. 3 and 4 except for the fact that FIGS. 3 and 4 actually shows the arrangement at the end of the tibial intramedullary canal preparation procedure.

In use, after the guide assembly 400 is positioned in the resected joint space 22 as shown in FIG. 6, the guide assembly 400 can be secured to the tibia by employing one or more fixation pins, such as k-wires or Steinmann pins. The guide head portion 420 can be provided with one or more holes extending therethrough for receiving such fixation pins. In the example shown in FIG. 6, the guide head portion 420 is provided with a plurality of holes 450; fixation pins 600 are shown inserted therethrough securing the guide assembly 400 to the tibia 6.

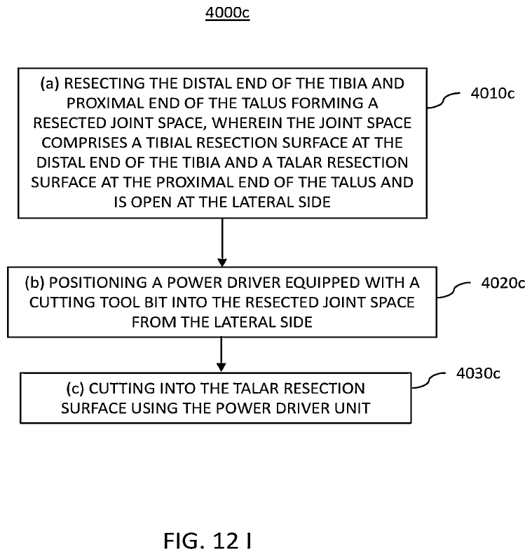

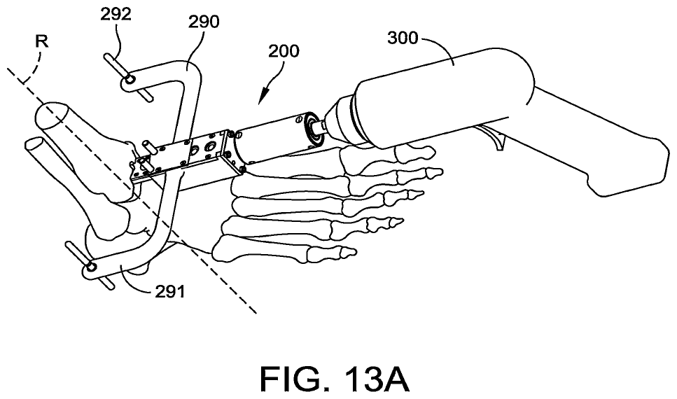

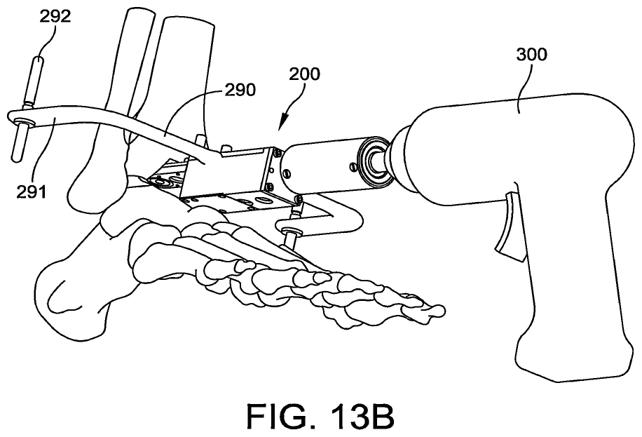

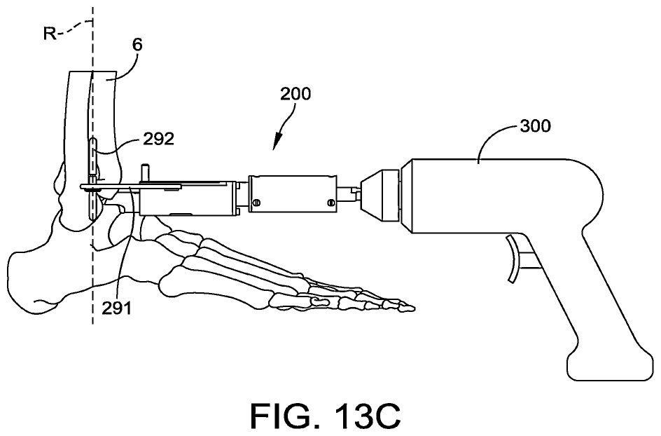

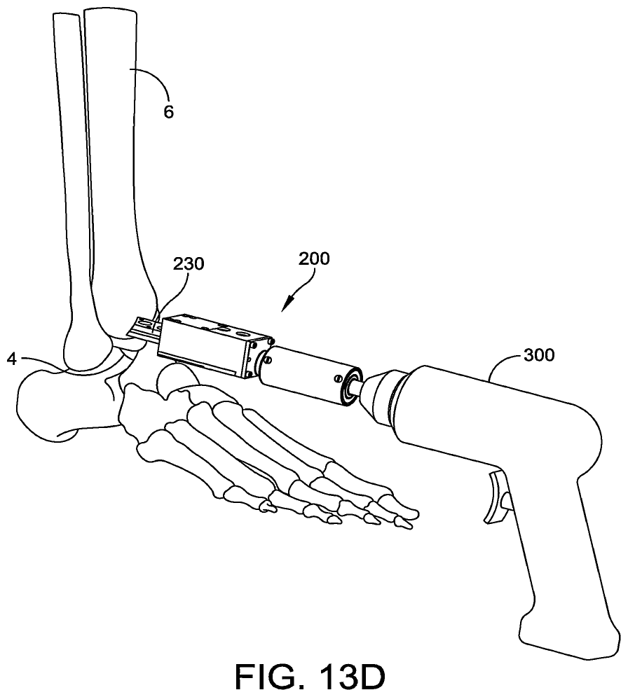

In some embodiments, the power driver adapter 200 can be aligned within the joint space 22 without the use of the alignment guide 400. For example, the power driver adapter 200 can be configured with an alignment arms 290 like the ones shown in FIGS. 13A-13C where the alignment arms 290 and the alignment pins 292 are radiopaque. The power driver adapter 200 can be inserted into the joint space 22 and viewed under a fluoroscopy to align the cutting tool bit 500. In some embodiments, the power driver adapter 200 can be made of radiolucent material and provided with radiopaque markers (a bullseye, for example) for alignment cues.

According to another aspect of the present disclosure, a surgical instrument kit is disclosed. The surgical instrument kit comprises a power driver adapter 200 configured for cutting into an intramedullary canal of a tibia, and one or more bone cutting tool bits (e.g. one or more reaming bits 500). The structure of the power driver adapter 200 is as described above. In some embodiments, the surgical instrument kit can also comprise a guide assembly 400 whose structure is as described above.

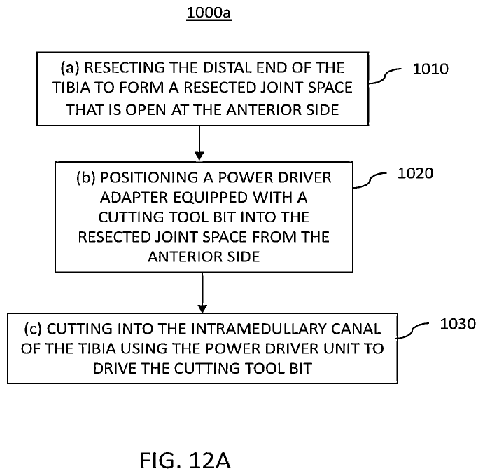

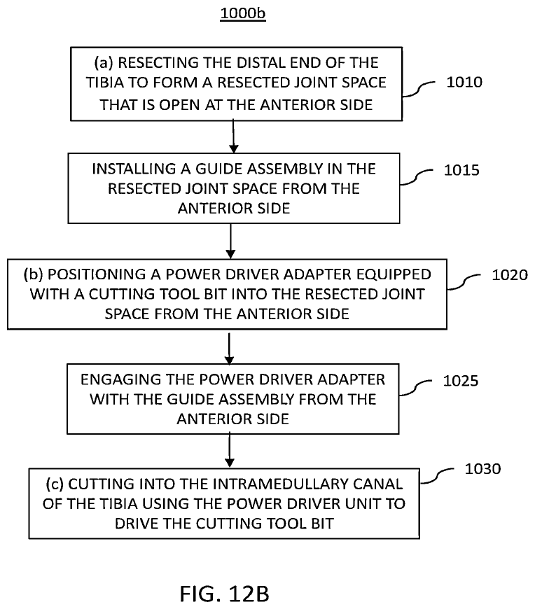

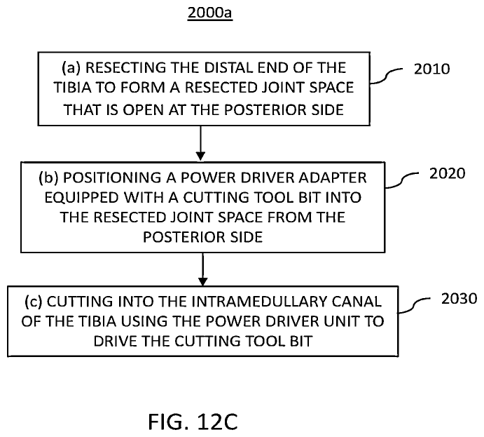

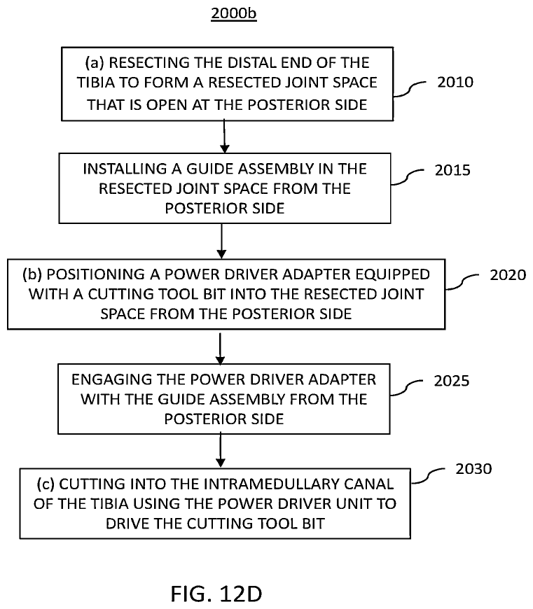

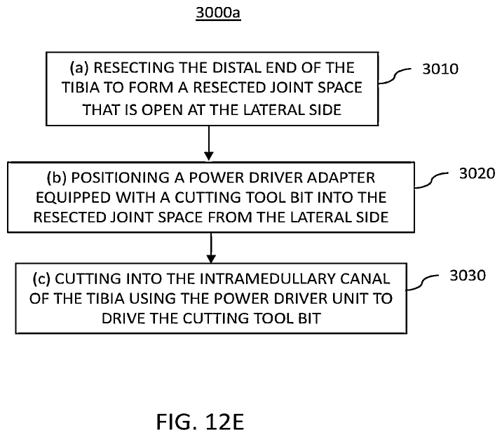

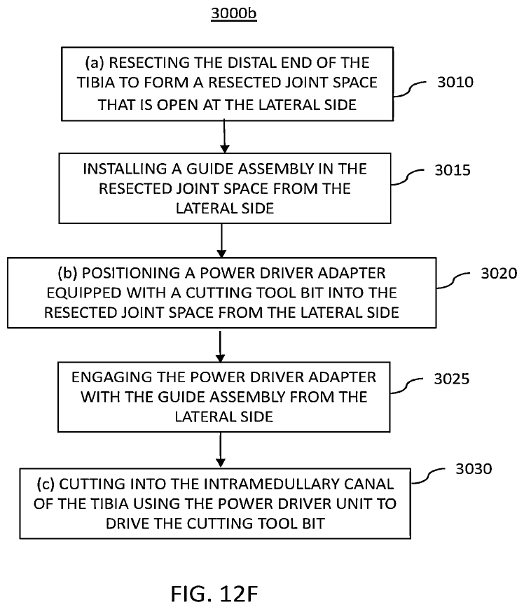

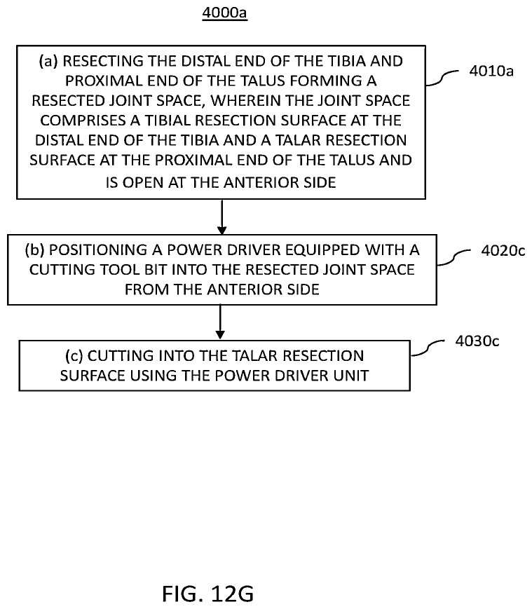

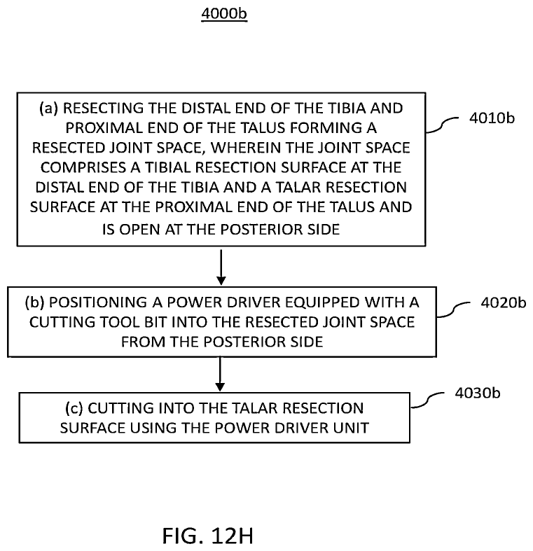

According to another aspect of the present disclosure, some methods for preparing the intramedullary canal in a tibia for receiving a tibial implant are disclosed. According to some embodiments, the flowchart 1000a in FIG. 12A in conjunction with FIGS. 13A-13C illustrates an example of such a method where the power driver adapter 200 equipped with a cutting tool bit 500 approaches the resected joint space 22 of an ankle from the anterior side for preparing the intramedullary canal in the distal end of the tibia for receiving a tibial implant according to an embodiment. The method comprises resecting the distal end of the tibia and forming a resected joint space 22 for receiving the tibial implant, where the joint space comprises a tibial resection surface at the distal end of the tibia and is open at the anterior side, see step 1010. If necessary, the proximal end of the talus may also need to be resected to properly form the resected joint space 22. Then, a power driver unit 300 with a power driver adapter 200 equipped with a cutting tool bit 500 is positioned into the joint space 22 from the anterior side, where the cutting tool bit 500 is aimed toward the intramedullary canal of the tibia, see step 1020. Next, the power driver unit 300 is turned on to drive the cutting tool bit 500 to cut into the intramedullary canal, see step 1030.

In some embodiments of the method, cutting into the intramedullary canal forms a void or a tibial cavity extending into the intramedullary canal for receiving a tibial stem or a tibial extension of an ankle replacement implant.

In the arrangement shown in FIGS. 13A-13C, an embodiment of the power driver adapter 200 that is provided with a pair of alignment arms 290 is shown. The alignment arms 290 assist with aligning the trajectory of the cutting tool bit 500 as the power driver adapter 200 is positioned into the resected joint space 22. The alignment arms 290 extend out from the power driver adapter 200 and have end portions 291 that curve around forward. The alignment arms 290 comprise of alignment posts 292 provided on the end portions 291 that represents the cutting trajectory of the cutting tool bit 500 and can be used to position the power driver adapter 200 visually while maintaining the alignment/trajectory of the cutting tool bit 500 before and during the cutting procedure. As shown in FIGS. 13A and 13C, the alignment arms 290 and the alignment posts 292 are configured so that the alignment posts 292 are parallel with the rotation axis R of the cutting tool bit 500 installed on the cutting tool bit receiving base 232 and the two alignment posts 292 are also in the same plane as the rotation axis R. This configuration allows the surgeon to use the alignment posts 292 as visual guides to align the power driver adapter 200 into the joint space 22. The side view in FIG. 13C shows the power driver adapter 200 in aligned position where the cutting tool bit's rotation axis R has been aligned with the intramedullary canal of the tibia 6 by using the alignment posts 292 as guides.

The flowchart 1000b in FIG. 12B in conjunction with FIGS. 3 and 4 illustrates another embodiment of the method in which a guide assembly 400 is used to align the trajectory of the cutting tool bit 500. The method illustrated in flowchart 1000b further comprises the step 1015 of installing a guide assembly 400 in the joint space from the anterior side of the ankle after the step 1010. Then, after positioning the power driver adapter 200 equipped with a cutting tool bit 500 into the resected joint space 22 from the anterior side in step 1020, one engages the power driver adapter 200 with the guide assembly 400 from the anterior side to align the position of the cutting tool bit 500, see step 1025. Next, the power driver unit 300 is turned on to drive the cutting tool bit 500 to cut into the intramedullary canal to prepare the intramedullary canal for a tibial implant, see step 1030.

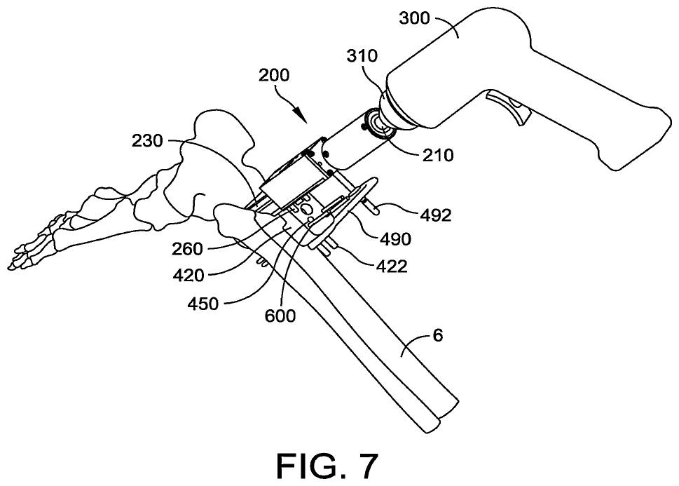

The flowchart 2000a in FIG. 12C in conjunction with FIG. 7 illustrates an example of a posterior approach method of preparing an intramedullary canal in a tibia for receiving a tibial implant according to an embodiment. The method comprises resecting the distal end of the tibia forming a resected joint space 22 for receiving the tibial implant, where the joint space comprises a tibial resection surface at the distal end of the tibia and is open at the posterior side, see step 2010. If necessary, the proximal end of the talus may also need to be resected to properly form the resected joint space 22. Then, a power driver adapter 200 equipped with a cutting tool bit 500 is positioned into the joint space 22 from the posterior side, where the cutting tool bit 500 is aimed toward the intramedullary canal of the tibia, see step 2020. Next, the power driver unit 300 is turned on to drive the cutting tool bit 500 to cut into the intramedullary canal to prepare the intramedullary canal for a tibial implant, see step 2030.

The flowchart 2000b in FIG. 12D illustrates another embodiment of the posterior approach method in which the method further comprises the step 2015 of installing a guide assembly 400 in the joint space from the posterior side of the ankle after the step 2010. Then, after positioning the power driver adapter 200 equipped with the cutting tool bit 500 into the resected joint space 22 from the posterior side in step 2020, one engages the power driver adapter 200 with the guide assembly 400 from the posterior side to align the position of the cutting tool bit 500, see step 2025. Next, the power driver unit 300 is turned on to drive the cutting tool bit 500 to cut into the intramedullary canal to prepare the intramedullary canal for a tibial implant, see step 2030.

FIG. 7 is an illustration of the posterior approach arrangement after the guide assembly 400 is positioned in the resected joint space 22. The guide head portion 420 is provided with a plurality of holes 450 for receiving one or more fixation pins 600 for securing the guide assembly 400 to the tibia 6 from the posterior side. The fixation pins 600 are shown inserted through the holes 450 securing the guide assembly 400 to the tibia 6. In this embodiment, the guide assembly 400 itself can include an alignment guide arm 490. The alignment guide arm 490 can be attached to the guide head portion 420 via an appropriate attachment mechanism. In the illustrated example, the guide head portion 420 is configured with one or more pins/screws 422 to which the alignment guide arm 490 is attached. The key feature of the alignment guide arm 490 is that it extends out sideways from the guide head portion 420 and is provided with an alignment post 492. This allows the operator/surgeon to visually align the guide assembly 400 during installation on to the tibia 6, which ensures that the cutting tool bit 500 will be properly aligned. The power driver adapter 200 equipped with the cutting tool bit 500 is then positioned into the joint space 22 from the posterior side of the patient and mated with the guide assembly 400. The alignment post 492 and the guide head portion 420 are configured so that when the power driver adapter 200 is engaged and aligned with the guide head portion 420 by slip-fitting the alignment pins 260 into the corresponding alignment slots 440 in the guide head portion 420, the alignment post 492 is in parallel relation to the rotational axis R of the cutting tool bit 500.

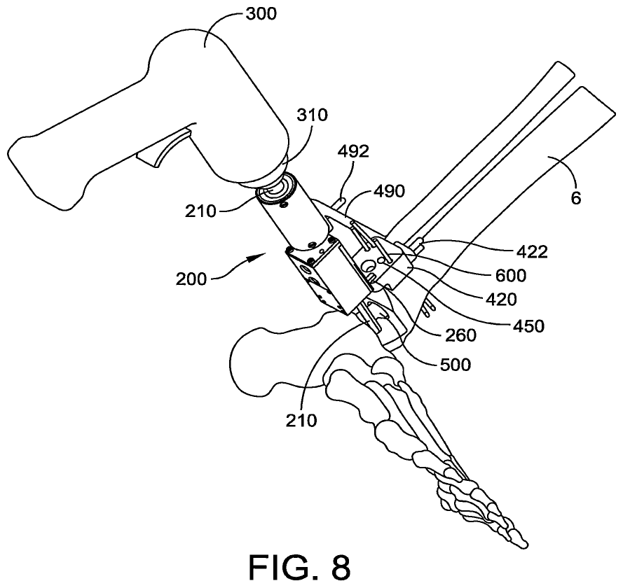

The flowchart 3000a in FIG. 12E in conjunction with FIG. 8 illustrates an example of a lateral approach method of preparing an intramedullary canal in a tibia for receiving a tibial implant according to an embodiment. The method comprises resecting the distal end of the tibia and, if necessary, the proximal end of the talus forming a resected joint space 22 for receiving the tibial implant, where the joint space comprises a tibial resection surface at the distal end of the tibia and is open at the lateral side, see step 3010. Then, the power driver adapter 200 equipped with the cutting tool bit 500 is positioned into the joint space 22 from the lateral side, where the cutting tool bit 500 is aimed toward the intramedullary canal of the tibia, see step 3020. Next, the power driver unit 300 is turned on to drive the cutting tool bit 500 to cut into the intramedullary canal to prepare the intramedullary canal for a tibial implant, see step 3030.

The flowchart 1000b in FIG. 12F illustrates another embodiment of the lateral approach method in which the method further comprises the step 3015 of installing a guide assembly 400 in the joint space from the lateral side of the ankle after the step 3010. Then, after positioning the power driver adapter 200 equipped with the cutting tool bit 500 into the resected joint space 22 from the lateral side in step 3020, one engages the power driver adapter 200 with the guide assembly 400 from the lateral side to align the position of the cutting tool bit 500, see step 3025. Next, the power driver unit 300 is turned on to drive the cutting tool bit 500 to cut into the intramedullary canal to prepare the intramedullary canal for a tibial implant, see step 3030.

FIG. 8 is an illustration of the lateral approach arrangement where the guide assembly 400 is then positioned in the resected joint space 22. The guide head portion 420 is provided with a plurality of holes 450 for receiving one or more fixation pins 600 for securing the guide assembly 400 to the tibia 6 from the posterior side. The fixation pins 600 are shown inserted through the holes 450 securing the guide assembly 400 to the tibia 6. The power driver adapter 200 equipped with the cutting tool bit 500 is then positioned into the joint space 22 from the lateral side of the patient and mated with the guide assembly 400. Similar to the description provided above in connection with FIG. 7, in some embodiments of the lateral approach arrangement, the guide assembly 400 can include an alignment guide arm 490 that is provided with an alignment post 492. The function of the alignment guide arm 490 and the alignment post 492 in this embodiment is similar to the embodiment shown in FIG. 7 and described.

The flowchart 4000a in FIG. 12G in conjunction with FIG. 13D illustrates an example of an anterior approach method of preparing the proximal end of a talus for receiving a talar implant according to an embodiment. The method comprises resecting the distal end of the tibia and, if necessary, the proximal end of the talus forming a resected joint space 22, where the joint space comprises a tibial resection surface at the distal end of the tibia and a talar resection surface at the proximal end of the talus and the joint space 22 is open at the anterior side, see step 4010. Then, the power driver adapter 200 equipped with the cutting tool bit 500 is positioned into the joint space 22 from the anterior side, where the cutting tool bit 500 is aimed toward the talus, see step 4020. Next, the power driver unit 300 is turned on driving the cutting tool bit 500 to cut into the talar resection surface and form a void extending into the talus for receiving a tarlar stem and/or augment for a talar implant, see step 4030.

The flowchart 4000b in FIG. 12H illustrates an example of a posterior approach method of preparing the proximal end of a talus for receiving a talar implant according to an embodiment. The method comprises resecting the distal end of the tibia and, if necessary, the proximal end of the talus forming a resected joint space 22, where the joint space comprises a tibial resection surface at the distal end of the tibia and a talar resection surface at the proximal end of the talus and the joint space 22 is open at the posterior side, see step 4010b. Then, the power driver adapter 200 equipped with the cutting tool bit 500 is positioned into the joint space 22 from the posterior side, where the cutting tool bit 500 is aimed toward the talus, see step 4020b. Next, the power driver unit 300 is turned on driving the cutting tool bit 500 to cut into the talar resection surface and form a void extending into the talus for receiving a talar stem and/or augment for a talar implant, see step 4030b.

The flowchart 4000c in FIG. 12I illustrates an example of a lateral approach method of preparing the proximal end of a talus for receiving a talar implant according to an embodiment. The method comprises resecting the distal end of the tibia and, if necessary, the proximal end of the talus forming a resected joint space 22, where the joint space comprises a tibial resection surface at the distal end of the tibia and a talar resection surface at the proximal end of the talus and the joint space 22 is open at the lateral side, see step 4010c. Then, the power driver adapter 200 equipped with the cutting tool bit 500 is positioned into the joint space 22 from the lateral side, where the cutting tool bit 500 is aimed toward the talus, see step 4020c. Next, the power driver unit 300 is turned on driving the cutting tool bit 500 to cut into the talar resection surface and form a void extending into the talus for receiving a talar stem and/or augment for a talar implant, see step 4030c.

In the various embodiments of the methods described herein, the power driver adapter 200 equipped with the cutting tool bit 500 can be positioned into that ankle joint space between the tibia and the talus before any resection cuts of the tibia or the talus are made. In such examples, an appropriately configured guide assembly jig (not shown) can be inserted into the joint between the tibia and the talus, then guide and position the cutting tool bit end of the power driver adapter 200 between the tibia and the talus before the resection cuts are made to the distal end of the tibia or the proximal end of the talus.

Although the devices, kits, systems, and methods have been described in terms of exemplary embodiments, they are not limited thereto. Rather, the appended claims should be construed broadly, to include other variants and embodiments of the devices, kits, systems, and methods, which may be made by those skilled in the art without departing from the scope and range of equivalents of the devices, kits, systems, and methods.

We claim:

1. A method of preparing an intramedullary canal in a tibia for receiving a tibial implant, wherein the tibia comprises a distal end, the method comprising:
a) resecting the distal end of the tibia forming a resected joint space for the tibial implant, wherein the joint space comprises a tibial resection surface at the distal end of the tibia and is open at an anterior side;
b) positioning a power driver, equipped with a cutting tool bit, into the resected joint space from the anterior side, wherein the cutting tool bit is aimed toward the intramedullary canal of the tibia; and
c) cutting into the intramedullary canal using the power driver.

2. The method of claim 1, further comprising:
installing a guide assembly in the joint space after step (a) but before step (b); and engaging the power driver with the guide assembly after the power driver is positioned into the joint space from the anterior side, wherein the engagement with the guide assembly aligns the cutting tool bit to be aimed toward the intramedullary canal of the tibia.

3. The method of claim 2, wherein installing the guide assembly in the joint space comprises:
inserting the guide assembly into the joint space from the anterior side;
aligning the guide assembly flush with the tibial resection surface;
securing the guide assembly to the tibia;
verifying the alignment and trajectory of the guide; and
securing the guide head portion to the tibia with one or more pins.

4. The method of claim 1, wherein cutting into the intramedullary canal forms a void or a tibial cavity extending into the intramedullary canal for receiving a tibial stem or a tibial extension of an ankle replacement implant.

5. A method of preparing an intramedullary canal in a tibia for receiving a tibial implant, wherein the tibia comprises a distal end, the method comprising:
a) resecting the distal end of the tibia forming a resected joint space for the tibial implant, wherein the joint space comprises a tibial resection surface at the distal end of the tibia and is open at posterior side;
b) positioning a power driver, equipped with a cutting tool bit, into the joint space from the posterior side, wherein the cutting tool bit is aimed toward the intramedullary canal of the tibia and configured to translate linearly in a direction that is (i) coaxial to a rotational axis of the cutting tool bit and (ii) orthogonal to a longitudinal axis of the cutting tool bit; and
c) cutting into the intramedullary canal using the power driver.

6. The method of claim 5, further comprising:
installing a guide assembly in the joint space after step (a) but before step (b); and engaging the power driver with the guide assembly after the power driver is positioned into the joint space from the posterior side, wherein the engagement with the guide assembly aligns the cutting tool bit to be aimed toward the intramedullary canal of the tibia.

7. The method of claim 6, wherein installing the guide assembly in the joint space comprises:
inserting the guide assembly into the joint space from the posterior side;
aligning the guide assembly flush with the tibial resection surface;
securing the guide assembly to the tibia;
verifying the alignment and trajectory of the guide; and
securing the guide head portion to the tibia with one or more pins.

8. The method of claim 5, wherein cutting into the intramedullary canal forms a void or a tibial cavity extending into the intramedullary canal for receiving a tibial stem or a tibial extension of an ankle replacement implant.

9. A method of preparing an intramedullary canal in a tibia for receiving a tibial implant, wherein the tibia comprises a distal end, the method comprising:
a) resecting the distal end of the tibia forming a resected joint space for the tibial implant, wherein the joint space comprises a tibial resection surface at the distal end of the tibia and is open at a lateral side;
b) positioning a power driver, equipped with a cutting tool bit, into the joint space from the lateral side, wherein the cutting tool bit is aimed toward the intramedullary canal of the tibia; and
c) cutting into the intramedullary canal using the power driver.

10. The method of claim 9, further comprising:
installing a guide assembly in the joint space after step (a) but before step (b); and engaging the power driver with the guide assembly after the power driver is positioned into the joint space from the lateral side, wherein the engagement with the guide assembly aligns the cutting tool bit to be aimed toward the intramedullary canal of the tibia.

11. The method of claim 10, wherein installing the guide assembly in the joint space comprises:
inserting the guide assembly into the joint space from the lateral side;
aligning the guide assembly flush with the tibial resection surface;
securing the guide assembly to the tibia;
verifying the alignment and trajectory of the guide; and
securing the guide head portion to the tibia with one or more pins.

12. The method of claim 9, wherein cutting into the intramedullary canal forms a void or a tibial cavity extending into the intramedullary canal for receiving a tibial stem or a tibial extension of an ankle replacement implant.

13. A power driver adapter configured for cutting into an intramedullary canal of a tibia, the power driver adapter comprising:
an elongated body having a driving end, a cutting tool bit receiving end, and a longitudinal axis;
wherein the driving end having a drive shaft coaxially located with the longitudinal axis and configured to mate with a power delivering unit that rotates the drive shaft coaxially about the longitudinal axis;
wherein the cutting tool bit receiving end comprises a cutting tool bit receiving base that is configured for engaging with a cutting tool bit and rotates the cutting tool bit for cutting action, wherein the cutting tool receiving base rotates with a rotational axis that is orthogonal to the longitudinal axis of the elongated body;
wherein the elongated body comprises a series of gears connecting the drive shaft to the cutting tool bit receiving end;
wherein the series of gears are configured in an arrangement that converts the coaxial rotation of the drive shaft to the rotation of the cutting tool bit receiving base.

14. The power driver adapter of claim 13, wherein the cutting tool bit is a reamer bit.

15. The power driver adapter of claim 13, wherein the cutting tool bit receiving base is configured to engage the threads of the cutting tool bit.

16. The power driver adapter of claim 13, wherein the cutting tool bit receiving base comprises a socket that engages with the cutting tool bit.

17. The power driver adapter of claim 13, wherein the series of gears comprises a bevel gear arrangement.

18. The power driver adapter of claim 13, wherein the series of gears comprises a spur gear arrangement.

19. The power driver adapter of claim 13, wherein the cutting tool bit receiving end of the elongated body is configured to translate linearly in a direction that is coaxial to the rotational axis of the cutting tool bit and orthogonal to the longitudinal axis when the cutting tool bit is being rotated.

20. The power driver adapter of claim 19, wherein the series of gears comprises a helical thread arrangement that translates the cutting tool bit receiving end linearly.

21. A method of preparing a talus for receiving a talar implant, wherein the talus comprises a proximal end, the method comprising:
(a) resecting the proximal end of the talus forming a resected joint space for the talar implant, wherein the joint space comprises a talar resection surface at the proximal end of the talus and is open at an anterior side;
(b) positioning a power driver, equipped with a cutting tool bit, into the resected joint space from the anterior side, wherein the cutting tool bit is aimed toward the talar resection surface; and
(c) cutting into the talar resection surface using the power driver.

22. The method of claim 21, wherein cutting into the talar resection surface forms a void that extends into the talus for receiving a talar stem and/or augment for a talar implant.

23. The method of claim 21, further comprising a step of resecting the distal end of the tibia and forming a resection joint space for the tibial implant before the step (a).

24. A method of preparing a talus for receiving a talar implant, wherein the talus comprises a proximal end, the method comprising:
(a) resecting the proximal end of the talus forming a resected joint space for the talar implant, wherein the joint space comprises a talar resection surface at the proximal end of the talus and is open at posterior side;
(b) positioning a power driver, equipped with a cutting tool bit, into the resected joint space from the posterior side, wherein the cutting tool bit is aimed toward the talar resection surface; and
(c) cutting into the talar resection surface using the power driver.

25. The method of claim 24, wherein cutting into the talar resection surface forms a void extending into the talus for receiving a talar stem and/or augment for a talar implant.

26. The method of claim 24, further comprising a step of resecting the distal end of the tibia and forming a resected joint space for the tibial implant before the step (a).

27. A method of preparing a talus for receiving a talar implant, wherein the talus comprises a proximal end, the method comprising:
(a) resecting the proximal end of the talus forming a resected joint space for the talar implant, wherein the joint space comprises a talar resection surface at the proximal end of the talus and is open at lateral side;
(b) positioning a power driver, equipped with a cutting tool bit, into the resected joint space from the lateral side, wherein the cutting tool bit is aimed toward the talar resection surface; and
(c) cutting into the talar resection surface using the power driver.

28. The method of claim 27, wherein cutting into the talar resection surface forms a void extending into the talus for receiving a talar stem and/or augment for a talar implant.

29. The method of claim 27, further comprising a step of resecting the distal end of the tibia and forming a resected joint space for the tibial implant before the step (a).

30. A method of preparing a talus for receiving a talar implant, wherein the talus comprises a proximal end, the method comprising:
(a) resecting the proximal end of the talus forming a resected joint space for the talar implant, wherein the joint space comprises a talar resection surface at the proximal end of the talus and is open at an anterior side;
(b) positioning a power driver, equipped with a cutting tool bit, into the resected joint space from the anterior side, wherein the cutting tool bit is aimed toward the talar resection surface and is configured to translate linearly in a direction that is (i) coaxial to a rotational axis of the cutting tool bit and (ii) orthogonal to a longitudinal axis of the cutting tool bit; and
(c) cutting into the talar resection surface using the power driver.

31. The method of claim 30, wherein cutting into the talar resection surface forms a void that extends into the talus for receiving a talar stem and/or augment for a talar implant.

32. The method of claim 30, further comprising a step of resecting the distal end of the tibia and forming a resection joint space for the tibial implant before the step (a).
